(19) United States
(12) Patent Application Publication
(10) Pub. No.: US 2026/0041487 A1
(43) Pub. Date: Feb. 12, 2026

(54) REGISTRATION AND NAVIGATION IN CRANIAL PROCEDURES

(71) Applicant: Medtronic Navigation, Inc., Lafayette, CO (US)

(72) Inventors: Manisha CHALAMALASETTI, Hyderabad, Telangana (IN); Arifmohamad H. MUJAWAR, Sangli, Maharashtra (IN); Pratima MEHTA, Gurugram, Haryana (IN); Jyothi S. BONTHU, Hyderabad, Telangana (IN); Praveena NARAYANABHATLA, Hyderabad, Telangana (IN)

(21) Appl. No.: 19/125,763

(22) PCT Filed: Oct. 30, 2023
(86) PCT No.: PCT/IB2023/060929

§ 371 Date: Apr. 30, 2025

Related U.S. Application Data
(60) Provisional application No. 63/381,930, filed on Nov. 2, 2022.

(51) Int. Cl. A61B 34/10; A61B 34/00; A61B 34/20; A61B 34/30; A61B 90/00

(52) U.S. Cl. CPC A61B 34/10; A61B 34/20; A61B 34/25; A61B 34/30; A61B 90/361; A61B 90/37; A61B 2034/105; A61B 2034/2055; A61B 2034/252; A61B 2034/305; A61B 2090/3612; A61B 2090/371; A61B 2090/374; A61B 2090/3762

(57) ABSTRACT

The disclosed systems, methods, and techniques perform an imaging registration and a cranial surgical procedure navigation. In one aspect, the systems, methods, and techniques use modality scans of the cranium of the patient, which are obtained preoperatively, to create a 3D image source. Then, using a machine vision system embedded in or on a movable elliptical mask attached to a robotic arm, the systems, methods, and techniques obtain surface imaging data of a face or a portion of the cranium of the patient while the patient is lying in a supine position atop an operating table. The surface imaging data are used to create a 3D image target. The 3D image target then is registered to the 3D image source to complete the imaging registration. Once the imaging registration is complete, a surgeon can perform an image-guided surgery of the cranium of the patient.

### BACKGROUND INFORMATION

In image-guided surgery for glioma removal, neurosurgeons usually plan the resection on images acquired before surgery and use the acquired images for guidance during the subsequent intervention. After the surgical procedure has begun, however, the preplanned images may become unreliable due to, for example, the brain shift phenomenon that may be caused by modifications of anatomical structures and imprecisions in the neuronavigational system.

To obtain an updated view of a resection cavity, a solution may be to collect intraoperative data, which can be acquired at different stages of the procedure in order to provide a better understanding of the resection. Such a solution may require re-registration of the images that are acquired intraoperatively. The re-registration of the intraoperative images may include manually placing tracers on points of interest of the anatomical part. Some algorithms may be utilized for tracer initialization, surface merge initialization, image-to-image registration, various calculations, etc.

### SUMMARY OF THE DISCLOSURE

The disclosed systems, methods, and techniques perform an imaging registration and a cranial surgical procedure navigation. The systems, methods, and techniques use modality scans of the cranium of the patient, which are obtained preoperatively, to create a 3D image source. The modality scans may be computerized tomography (CT) scans, magnetic resonance imaging (MRI) scans, positron emission tomography (PET) scans, single photon emission computed tomography (SPECT) scans, or a combination thereof. Then, using a machine vision system embedded in or on a movable elliptical mask attached to a robotic arm, the systems, methods, and techniques obtain surface imaging data of a face or a portion of the cranium of the patient while the patient is lying in a supine position atop an operating table. To capture the surface imaging data, the machine vision system utilizes at least one imaging sensor, at least one infrared sensor, or a combination thereof. The surface imaging data are used to create a 3D image target. The 3D image target then is registered to the 3D image source to complete the imaging registration. Once the imaging registration is complete, a surgeon can perform an image-guided surgery of the cranium of the patient.

In one aspect, a method for performing an imaging registration and a cranial surgical procedure navigation includes obtaining modality scans of a cranium of a patient. The method also includes creating, using the modality scans, a first 3D model of the cranium of the patient. The method also includes obtaining, using a movable elliptical mask attached to a robotic arm, surface imaging data of a face or a portion of the cranium of the patient while the patient is lying in a supine position atop an operating table. The robotic arm gradually moves the elliptical mask approximately 180 degrees around the face or the portion of the cranium of the patient in a circular motion from: i) a first position to a second position, where the first position includes the elliptical mask approximately facing the nose of the patient, and the second position includes the elliptical mask approximately facing an ear of the patient; ii) the second position to a third position, where the third position includes the elliptical mask approximately facing another ear of the patient; and iii) the third position to the first position. The method also includes creating, using the surface imaging data, a second 3D model of the face or the portion of the cranium of the patient. The method also includes registering the second 3D model to the first 3D model. The method also includes guiding a physician during a cranial surgical procedure by displaying the first 3D model, the second 3D model, or a combination thereof on a display screen.

In another aspect, a computing apparatus with at least a processor and a computer-readable medium storing instructions that, when executed by the processor, configure the apparatus to perform the above-mentioned method.

In some embodiments, the computer-readable medium may be and/or include any suitable data storage media, such as volatile memory and/or non-volatile memory. Examples of volatile memory may include a random-access memory (RAM), such as a static RAM (SRAM), a dynamic RAM (DRAM), or a combination thereof. Examples of non-volatile memory may include a read-only memory (ROM), a flash memory (e.g., NAND flash memory, NOR flash memory), a magnetic storage medium, an optical medium, a ferroelectric RAM (FeRAM), a resistive RAM (RRAM), and so forth. Moreover, the computer-readable medium does not include transitory propagating signals or carrier waves.

In some embodiments, the processor may be substantially any electronic device that may be capable of processing, receiving, and/or transmitting the instructions that may be included in, permanently or temporarily saved on, and/or accessed by the computer-readable medium. The processor may be implemented using one or more processors (e.g., a central processing unit (CPU), a graphic processing unit (GPU)), and/or other circuitry, where the other circuitry may include at least one or more of an application specific integrated circuit (ASIC), a field programmable gate array (ASIC), a microprocessor, a microcomputer, and/or the like. Furthermore, the processor may be configured to execute the instructions in parallel, locally, and/or across a network by, for example, using cloud and/or server computing resources.

In one aspect, a system for image registration for cranial surgical procedures includes a movable machine vision system mountable to a robotic arm and configured to obtain surface imaging data of a face or a portion of a cranium of a patient. The system also includes a fixture holding the cranium, where the fixture includes a first, a second, and a third arm to collectively define a size and a position of the cranium by utilizing respective reflective spheres visible or trackable by the movable machine vision system. The system also includes a console configured to create a first 3D model of the face or the portion of the cranium using the surface imaging data and register the first 3D model to a second 3D model. The second 3D model is created using preoperative medical imaging data, such as CT scans, MRI scans, PET scans, SPECT scans, or a combination thereof. The system includes a display configured to present to a physician the first 3D model, the second 3D model, or a combination thereof during the cranial surgical procedure.

This disclosure describes systems, methods, and techniques to reduce the need of, or obviate, a manual registration and to enhance the accuracy of the registration. Accordingly, embodiments described in this disclosure can be incorporated in the above-mentioned processes for cranial surgical procedures.

Other technical features may be readily apparent to one skilled in the art from the following figures, descriptions, and claims.

Additional aspects and advantages will be apparent from the following detailed description of embodiments, which proceeds with reference to the accompanying drawings.

### BRIEF DESCRIPTION OF THE DRAWINGS

FIG. 1 shows an environment of a system for performing an imaging registration and a cranial surgical procedure navigation, in accordance with examples described herein.

FIG. 2 shows an example drawing of a side-elevation view of an adjustable three-arm fixture and an elliptical mask, where the adjustable three-arm fixture and the elliptical mask are used during the imaging registration and/or cranial surgical procedure navigation, in accordance with examples described herein.

FIG. 3 shows an example drawing of a patient-facing view of the elliptical mask, where the elliptical mask includes various sensors that are used during the imaging registration and/or the cranial surgical procedure navigation, in accordance with examples described herein.

FIG. 4 shows an example drawing that includes facial landmarks of a patient's face, where the facial landmarks are used to determine various features of the patient's face, in accordance with examples described herein.

FIG. 5 shows an environment with a display screen displaying images of a patient's cranium acquired using one of various possible modality scans, in accordance with examples described herein.

FIG. 6 illustrates a block diagram of an example method for performing an imaging registration and a cranial surgical procedure navigation, in accordance with examples described herein.

FIG. 7 illustrates a block diagram with additional details of the example method of FIG. 6, in accordance with examples described herein.

FIG. 8 illustrates a block diagram of a method for matching features of a 3D image target to the same features of the 3D image source, in accordance with examples described herein.

### DETAILED DESCRIPTION OF EMBODIMENTS

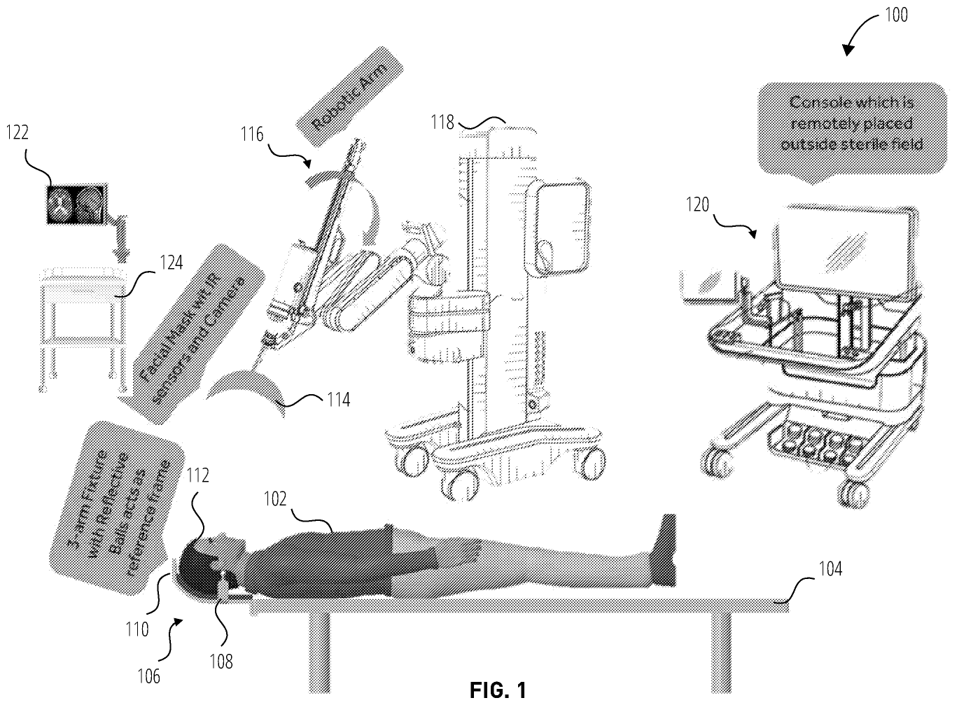

FIG. 1 illustrates an environment of a system 100 for performing an imaging registration and a cranial surgical procedure navigation, in accordance with examples described herein. In the example of FIG. 1, a patient 102 is lying in a supine position atop an operating table 104 (“table 104”) equipped with an adjustable three-arm fixture 106. The side-elevation view of FIG. 1 illustrates a first lateral arm 108 and a central arm 110 of the adjustable three-arm fixture 106. A second lateral arm of the adjustable three-arm fixture 106 is not explicitly illustrated due to being obscured by a cranium 112 of the patient 102. The cranium 112 of the patient 102 is placed in an opening of the adjustable three-arm fixture 106.

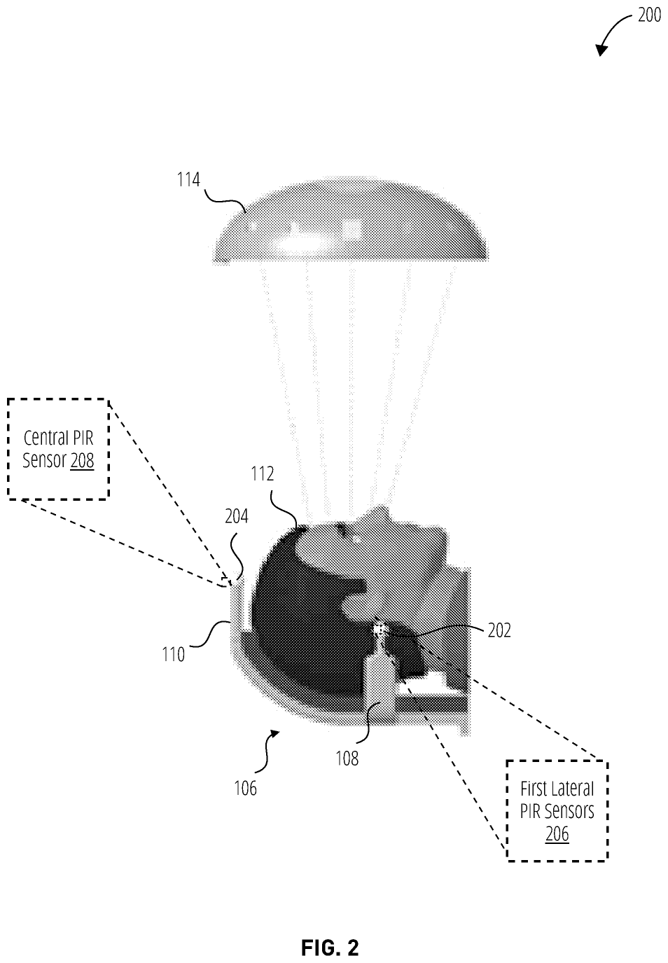

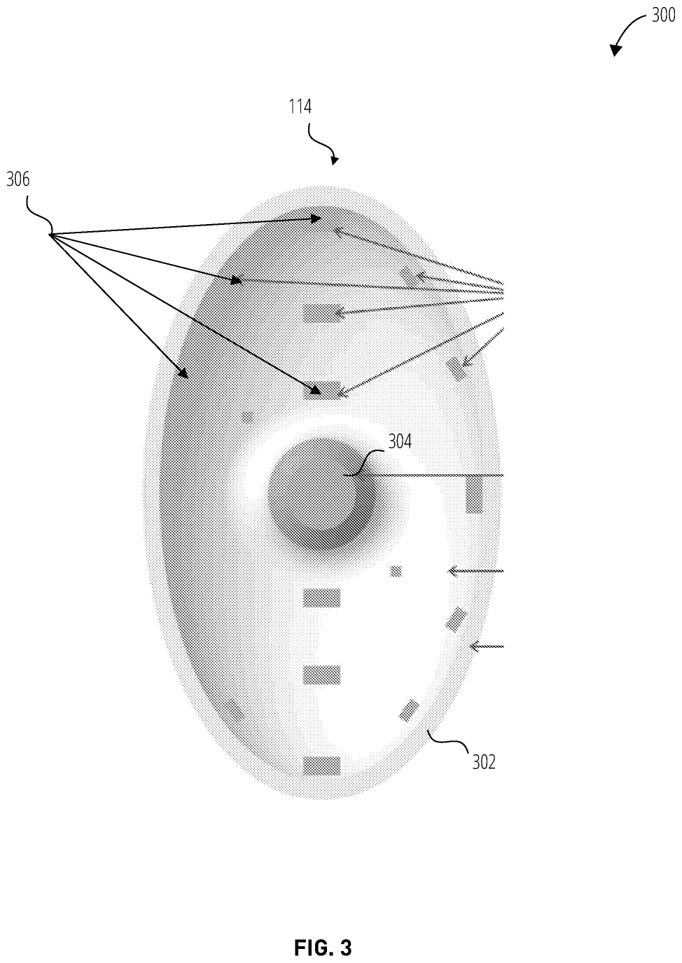

This disclosure focuses on the system 100 that utilizes the adjustable three-arm fixture 106, since the adjustable three-arm fixture 106 can effectively determine, hold firm, and/or continuously monitor the position of the cranium 112 of the patient 102. The system 100 also utilizes an elliptical mask 114 that is mounted on a robotic arm 116, and the elliptical mask 114 is further described, partly, with reference to FIG. 2 and FIG. 3. As is illustrated in FIG. 1, the robotic arm 116 is mounted on a cart 118, and the cart 118 is mobile and may be positioned anywhere in an operating room. In some embodiments, the robotic arm 116 is controllable from a remote location using a console 120, and the console 120 may be located outside the operating room, partly, to reduce the risk of contaminating the operating room and/or save some of the space of the operating room.

In some embodiments, the imaging registration enables a medical professional (not illustrated) to perform an image-guided surgery (IGS) by displaying a portion of or the whole cranium 112 on a display screen 122. In some embodiments, the display screen 122 can be mounted on a second cart 124, which may be located inside the operating room. Additionally, or alternatively, the system 100 may utilize a communication network to display the image(s) displayed on the display screen 122 on another display screen (e.g., a display screen of the console 120), which may be located outside the operating room.

The system 100 is designed for a specific anatomical part of a patient, namely, the cranium (e.g., the cranium 112 of the patient 102). Nevertheless, the system 100 may be modified to use other adjustable fixtures and/or differently shaped masks. For example, the system may be modified to use an adjustable greater-than-three-arms fixture, such as an adjustable five-arm fixture. As another example, an elliptical-shaped mask may be advantageous for a cranium-related procedure, but another shape may be more advantageous for another anatomical part of the patient. Therefore, the systems, methods, and techniques described herein may be modified to perform image registrations and surgical procedure navigations of other-than-cranial surgical procedures.

FIG. 2 shows an example drawing of a side-elevation view 200 of the adjustable three-arm fixture 106 and the elliptical mask 114, where the adjustable three-arm fixture 106 and the elliptical mask 114 are used in the imaging registration and/or the cranial surgical procedure navigation, in accordance with examples described herein. Note that FIG. 2 is illustrated and described in the context of FIG. 1. Moreover, FIG. 2 may include one or more reference numbers of FIG. 1.

As is illustrated, the adjustable three-arm fixture 106 includes the first lateral arm 108, the central arm 110, and the second lateral arm (obscured by the cranium 112 of the patient 102). The adjustable three-arm fixture 106 may be mechanically and/or electromechanically adjusted depending on the size of the cranium 112. Each arm of the adjustable three-arm fixture 106 includes a reflective ball or sphere. Specifically, the first lateral arm 108 includes a first lateral reflective ball 202; the central arm 110 includes a central reflective ball 204; and the second lateral arm (not illustrated) includes a second lateral reflective ball (not illustrated). Each reflective ball includes a passive infrared (PIR) sensor that may be embedded in (or on) the reflective balls. For clarity, as is illustrated with the dashed lines, a first lateral PIR sensor 206 may be embedded inside the first lateral reflective ball 202; a central PIR sensor 208 may be embedded inside the central reflective ball 204; and a second lateral PIR sensor (not illustrated) may be embedded inside the second lateral reflective ball (not illustrated).

In some embodiments, each PIR sensor includes a transmitter and a receiver of, for example, infrared (IR) light. The transmitter of a PIR sensor may transmit the IR light (e.g., omnidirectionally), and the receiver receives a reflected IR light off the cranium 112 of the patient 102. Consequently, each PIR sensor determines the distance of a respective arm of the adjustable three-arm fixture 106 and/or of a respective reflective ball from the cranium 112 of the patient 102. Based on the distances determined by the PIR sensor, in some embodiments, the system 100 (or a component thereof) can automatically (e.g., electromechanically) adjust each arm of the adjustable three-arm fixture 106 around the cranium 112 according to a predetermined configuration. In aspects, the predetermined configuration may be based on the size and/or dimensions of the cranium 112 of the patient 102. In other aspects, the predetermined configuration may be established preoperatively based on the modality scans and/or the 3D image source. Each PIR sensor aids the system 100 to adjust each arm of the adjustable three-arm fixture 106 to be within a range distance from the cranium 112 (head), where the range distance may include a low threshold distance (e.g., 0.2 mm) and a high threshold distance (e.g., 1.0 mm) from the cranium 112. The adjustable three-arm fixture 106 can be adjusted to accommodate the head of an infant patient, the head of an adult patient, and/or any other head size.

Additionally, or alternatively, the PIR sensors may help guide a medical professional to manually (e.g., mechanically) adjust each arm of the adjustable three-arm fixture 106 around the cranium 112 of the patient 102. For example, the medical professional may manually adjust each arm of the adjustable three-arm fixture 106, and the system may include a speaker and/or a display screen to guide the medical professional using acoustic beeps, alarms, and/or phrases, such as “move the arm closer to the patient's cranium,” “you are outside the range distance,” and/or other phrases of such effect. Additionally, or alternatively, the system 100 and/or the adjustable three-arm fixture 106 may utilize proximity sensors using other sensor technologies (e.g., radar technology, etc.) to determine the distance of each arm of the adjustable three-arm fixture 106 and/or each reflective ball from the cranium 112 of the patient 102.

In some aspects, the three reflective balls may be in continuous communication with a camera(s) (not illustrated in FIG. 2) of the elliptical mask 114, and the camera(s) of the elliptical mask 114 can track its position relative to the three reflective balls. In other aspects, the three reflective balls (with the aid of their respective PIR sensors) can create a reference frame of the adjustable three-arm fixture 106 and/or the cranium 112 of the patient 102 by, for example, determining and/or continuously monitoring the position of the cranium 112 during the imaging registration and a cranial surgical procedure navigation. Note that each PIR sensor can establish an x-y-z coordinate in space. Based on the three different x-y-z coordinates, the system 100 can define the position of the reflective balls, each arm of the adjustable three-arm fixture 106, and/or the cranium 112 of the patient 102. In other aspects, the reflective balls aid the system 100 map the live anatomy (e.g., the cranium 112) of the patient 102 to a three-dimensional (3D) model of the anatomy (e.g., the 3D image source).

FIG. 3 shows an example drawing of a patient-facing view 300 of the elliptical mask 114, where the elliptical mask 114 includes various sensors that are used during the imaging registration and/or the cranial surgical procedure navigation, in accordance with examples described herein. FIG. 3 is illustrated and described in reference to FIGS. 1 and 2, and FIG. 3 may include one or more reference numbers of FIGS. 1 and 2.

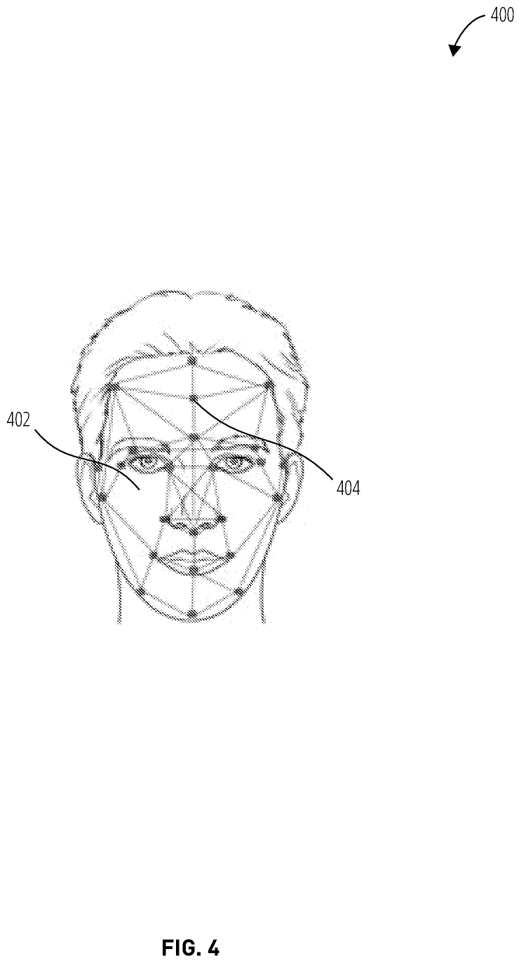

The elliptical mask 114 is constructed using a material that is suitable and/or graded for an operating room environment. For example, the elliptical mask 114 may be constructed using a medical-grade polycarbonate material. The elliptical mask 114 includes at least one light source. In some embodiments, the light source may be a light-emitting diode (LED) 302 that may be encapsulated in a medical-grade silicone frame. The LED 302 may continuously illuminate the reflective balls of the adjustable three-arm fixture 106 and the face 402 in FIG. 4 of the patient 102 during the imaging registration and/or the cranial surgical procedure navigation.

In some embodiments, the various sensors of the elliptical mask 114 that are used utilized during the imaging registration and/or the cranial surgical procedure navigation may be incorporated in a machine vision system (or a multi-camera system). The machine vision system may utilize one or more optical (e.g., visible) light digital camera(s) 304, such as one or more red-green-blue (RGB) cameras.

In some embodiments, the camera(s) 304 of the elliptical mask 114 continuously monitor the reflective balls of the adjustable three-arm fixture 106. By so doing, the camera(s) 304 of the elliptical mask 114 can track its position relative to the three reflective balls, the adjustable three-arm fixture 106, and/or the live anatomy (e.g., cranium 112) during the imaging registration and/or during the cranial surgical procedure.

In some embodiments, the camera(s) 304 may use a variety of technologies that are capable of capturing high-resolution images and converting the visible light into electrical signals. Such camera technologies may include complementary metal-oxide-semiconductor (CMOS) cameras, charge-coupled device (CCD) cameras, or another camera (or sensor) technology. The resolution of the camera(s) 304 may depend on a working distance, a field-of-view (FOV), the count of physical pixels in the camera(s) 304's sensor(s), and/or other factors and/or parameters. Regardless of the camera technology, the image sensors of the camera(s) 304 can capture a large array of pixels of an image with considerable physical detail, such as dimensions, edge location, movement, relative position, color information, and/or so forth. The machine vision system may utilize a digitizing device (e.g., a frame grabber) that translates (e.g., converts) the images captured by the camera(s) 304 into a digital output.

Although not explicitly illustrated in FIG. 3, in some embodiments, the camera(s) 304 may include and utilize two image sensors (e.g., two CMOS images sensors, two CCD image sensors) that may be separated by a minimum distance (e.g., 1 cm, 2 cm, 5 cm, or another distance). In such a case, the camera(s) 304 may be capable of capturing a stereo vision, and the machine vision system can perform 3D measurements having a depth perception (e.g., similar to two eyes). The two image sensors of the camera(s) 304 can capture a target, where the target may be a 3D image target of the patient 102's face and/or a portion of the patient 102's cranium 112, such as the portion of the cranium 112 facing the elliptical mask 114.

As is illustrated in FIG. 3, the elliptical mask 114 also includes one or more IR sensors 306 (“IR sensor(s) 306”) that may be embedded at various strategic locations in or on the elliptical mask 114. In some embodiments, each of the IR sensor(s) 306 includes an IR light transmitter (e.g., an IR LED) and an IR receiver. The IR sensor(s) 306 are utilized by a navigation tracker, where the navigation tracker is an IR tracker. The navigation tracker may utilize the IR sensor(s) 306 to continuously track the position of various medical instruments (not illustrated) used by a medical professional (e.g., a surgeon) during the cranial surgical procedure. To do so, each of the medical instruments may include one or more reflective balls embedded at one or more locations of the medical instruments. Furthermore, the IR sensor(s) 306 may aid the medical professional to track the position(s) of the medical instruments relative to the live anatomical part (e.g., the cranium 112), the 3D image target, and/or the preoperative 3D image source. It is to be appreciated that the elliptical mask 114 obviates a need for another enclosure associated solely with the navigation tracker.

Moreover, since the IR sensor(s) 306 can effectively and/or accurately be used to determine depth information of an image (or video), in addition to the camera(s) 304, the machine vision system may also utilize one, some, or all of the IR sensor(s) 306. Therefore, in some embodiments, the IR sensor(s) 306 of the elliptical mask 114 may be utilized by the navigation tracker and the machine vision system. In a case where the machine vision system utilizes the camera(s) 304 and one, some, or all of the IR sensor(s) 306 to capture images, the captured images may be referred to as RGB and IR depth (RGBIRD) images.

Since, in some embodiments, the machine vision system may utilize more than one image sensor technology, the machine vision system may utilize an auto-exposure (AE) algorithm to synchronize in real time an AE output, frame length times, frame-per-second frequencies, and/or the frame length lines of the RGB cameras (e.g., camera(s) 304) and the IR sensors (e.g., IR sensor(s) 306) to enhance the RGBIRD image. Generally, the duty cycles of the RGB sensors are greater (longer) than the duty cycles of the IR sensors. The machine vision system, however, can synchronize the timing of each IR sensor to match the timing of the RGB sensors. Furthermore, the machine vision system can align the active portion(s) of the duty cycle(s) of the IR sensors to fall within the active portion of the duty cycle(s) of the RGB sensors. By so doing, the machine vision system can capture RGB information (e.g., color, width, length) and IR depth information of the same image frame(s). Consequently, the machine vision system and/or the elliptical mask 114 can accurately capture a 3D image (e.g., a 3D image target) of the patient 102's face and/or of a portion of the patient 102's cranium 112 during imaging registration and/or cranial surgical procedure.

FIG. 4 shows an example drawing 400 that includes facial landmarks 404 (e.g., psychometric points) of a patient's face 402, where the facial landmarks 404 are used to determine various features of the patient's face 402, in accordance with examples described herein. FIG. 4 is described in the context of FIGS. 1, 2, and 3.

The x-y-z coordinates of each of the facial landmarks 404 of the patient's face 402 are unique regarding the patient 102. The x-y coordinates of the facial landmarks 404 may define a distance between a first specific facial landmark to a second specific facial landmark, and the distance between the two specific facial landmarks is also unique regarding the patient 102. For example, the facial landmarks 404 may define a distance between the most inner parts of the two eyebrows; a distance between the most outer parts of the two eyebrows; a distance between the two irises (if the patient is awake); a distance between the centers of the two eyes (if the patient has their eyes closed); a distance between the two ears; a distance between the two jaw lines (bones); a distance between the two zygomatic bones; a distance between the two temporal bones; a width of the lips; a width of the nose; a width of the head; and/or other features that may not be explicitly illustrated and/or described herein. Moreover, the facial landmarks 404 (e.g., the psychometric points) include depth information of the face 402 and/or the cranium 112 of the patient 102. The various facial landmarks 404 may have different depths (e.g., z coordinates) and may aid the system 100 and/or a component thereof (e.g., the machine vision system) to determine topographic-like information of the face 402 and/or of a portion of the cranium 112 of the patient 102 to perform an imaging registration and a cranial surgical procedure navigation.

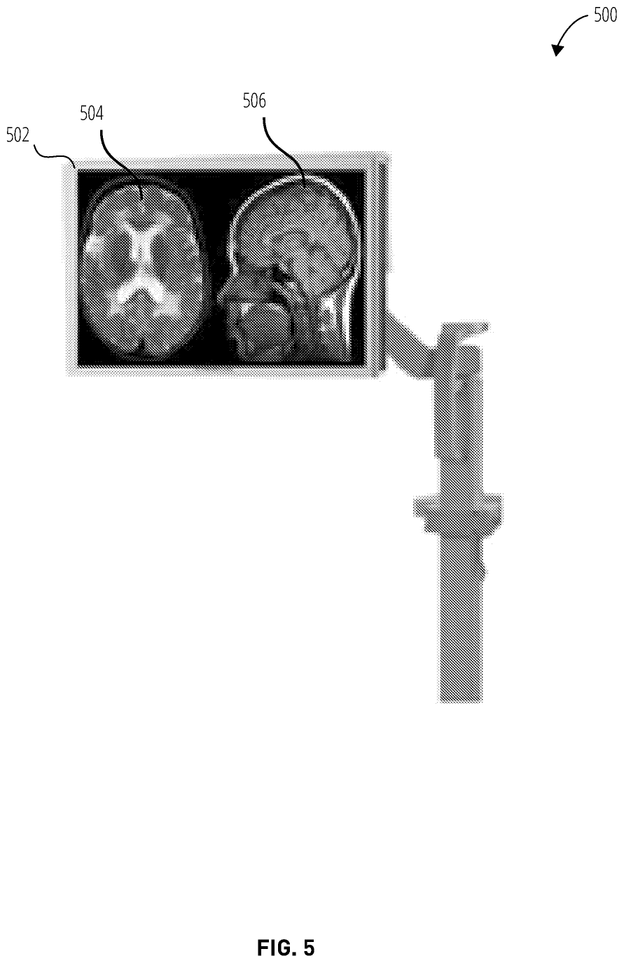

FIG. 5 shows an environment 500 with a display screen 502 displaying examples of a first image 504 and a second image 506 of a patient's cranium 112, where the first image 504 and the second image 506 are acquired using one of various modality scans, in accordance with examples described herein. The display screen 502 of FIG. 5 is the same as or equivalent to the display screen 122 of FIG. 1.

The first image 504 and the second image 506 are CT scans of the cranium 112 of the patient 102. However, a medical professional may use a variety of modality scans, such as CT scans, MRI scans, PET scans, SPECT scans, or a combination thereof (e.g., CT-PET scans).

The modality scans are acquired preoperatively using a respective scanner (not illustrated), such as a CT scanner, an MRI scanner, a PET scanner, a SPECT scanner, a CT-PET scanner, or using more than one type of scanner. Based, on the modality scans, using an application software, a medical professional (e.g., a technician, an engineer, a surgeon) may preoperatively create a 3D model of the cranium 112, where the 3D model of the cranium 112 is a contour (e.g., an outline) of the cranium 112. The 3D model of the cranium 112 may be referred to herein as a 3D image source.

In some embodiments, the 3D image source is a reference to a 3D image target during the imaging registration and/or the cranial surgical procedure navigation, as is further described herein. Furthermore, the 3D image source is a reference to various instruments used by a medical professional during, for example, the cranial surgical procedure.

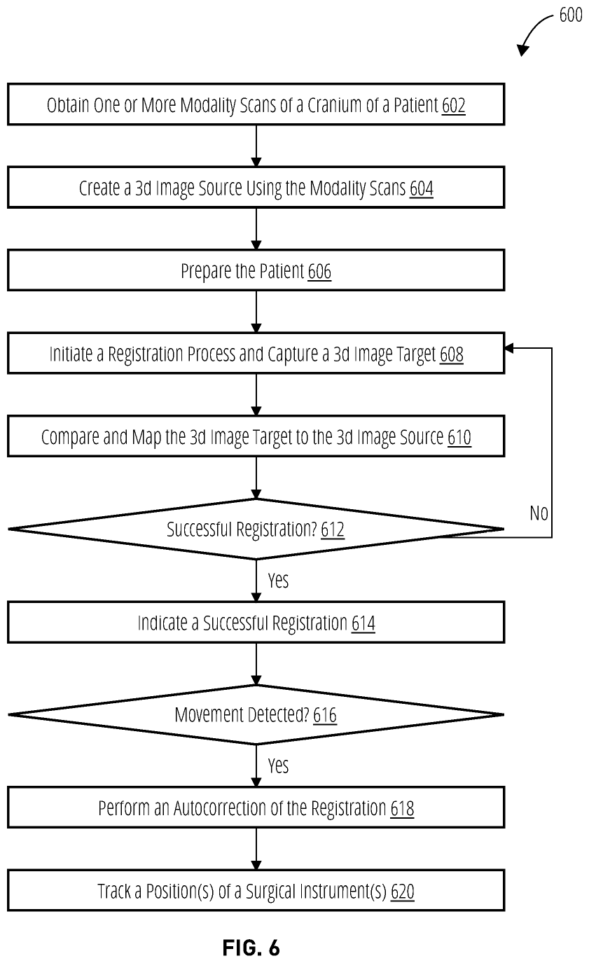

FIG. 6 illustrates a block diagram 600 of an example method for performing an imaging registration and a cranial surgical procedure navigation, in accordance with examples described herein. FIG. 6 is partly described in the context of FIGS. 1, 2, 3, 4, and 5; and FIG. 6 may include one or more reference numbers of FIGS. 1, 2, 3, 4, and 5. The steps of the method do not necessarily need to be executed in any specific order, or even sequentially, nor need the steps be executed only once, unless otherwise specified. Furthermore, the method can be utilized by using one, more than one, and/or all the steps that are illustrated in FIG. 6.

Therefore, the method does not necessarily include a minimum, an optimum, or a maximum number of steps that are needed to implement the systems, methods, and techniques described herein.

At step 602, the method may include obtaining one or more modality scans of the cranium 112 of the patient 102. As discussed, the modality scans may include CT scans, MRI scans, PET scans, SPECT scans, or a combination thereof (e.g., CT-PET scans). For clarity, step 602 of the method is executed and/or completed preoperatively.

At step 604, the method may include uploading the modality scans onto the system 100 (or a component thereof) to create a 3D model and/or a 3D image source of the cranium 112.

To create the 3D model and/or the 3D image source the method includes converting the two-dimensional (2D) images (e.g., the modality scans) into a 3D image (e.g., the 3D model, the 3D image source) using one or more techniques, such as a volume rendering technique. For example, the 2D images may be represented using a first file format, such as a digital imaging and communications in medicine (DICOM) file format, and the 3D image (e.g., the 3D model) may be represented using a second file format, such as a point cloud or a 3D model. In aspects, a volumetric pixel in a 3D space (e.g., a voxel) may be a function of the size of a 2D pixel, where the size of the 2D pixel may be a width along a first axis (e.g., x-axis) and a height along a second axis (e.g., y-axis). For clarity, step 604 of the method is executed and/or completed preoperatively.

At step 606, the method includes the medical professionals preparing the patient 102. In some embodiments, the preparation of the patient 102 may include laying down the patient 102 on the table 104; placing the cranium 112 of the patient 102 inside the adjustable three-arm fixture 106; placing the elliptical mask 114 in a position facing the face 402 of the patient 102; holding the patient 102's cranium 112 in a firm position using the adjustable three-arm fixture 106; and manually (e.g., mechanically) or automatically (e.g., electromechanically) adjusting each arm of the adjustable three-arm fixture 106 around the cranium 112, as is partly described with reference to FIGS. 1 and 2.

At step 608, a medical professional may initiate a registration process by, for example, entering a command, pressing a tab on a display screen, clicking a mouse, or utilizing any other user interface remotely (e.g., using the console 120) or inside the operating room (e.g., using the cart 118). The camera(s) 304 of the elliptical mask 114 may focus on the reflective balls of the adjustable three-arm fixture 106 and determine the positions of the reflective balls. After the camera(s) 304 of the elliptical mask 114 determine the position of the reflective balls of the adjustable three-arm fixture 106, the robotic arm 116 positions the elliptical mask 114 directly above the cranium 112 of the patient 102 and facing the face 402 of the patient 102. The robotic arm 116 moves (or rotates) the elliptical mask 114 approximately 180 degrees (e.g., +90 degrees and −90 degrees) to capture the features of the face 402 and/or at least a portion of the cranium 112 of the patient 102 using the camera(s) 304 and/or the IR sensor(s) 306 of the elliptical mask 114. In addition to the camera(s) 304, in some embodiments, the elliptical mask 114 may also utilize one, some, or all of the IR sensor(s) 306 to capture the features of the face 402 and/or at least a portion of the cranium 112 of the patient 102.

In some embodiments, the robotic arm 116 may reference three positions: a first position, a second position, and a third position. The first position may be a neutral position, or a zero degrees position, where the robotic arm 116 may position the elliptical mask 114 directly above (and facing) the face 402 of the patient 102. In the neutral position, the camera(s) 304 of the elliptical mask 114 may be equidistant from the first lateral reflective ball 202 of FIG. 2 and the second lateral reflective ball (not illustrated).

In some embodiments, the second position may be a first lateral position or a +90 degrees position, and the second position may be closest to the first lateral reflective ball 202. In aspects, the robotic arm 116 gradually moves (or rotates) the elliptical mask 114 from the neutral position (e.g., zero degrees) to the second position, while continuously capturing images of the face 402 of the patient 102 using the camera(s) 304 and/or the IR sensor(s) 306 embedded in or on the elliptical mask 114. In some embodiments, in the second position, the camera(s) 304 of the elliptical mask 114 is approximately or directly facing and capturing the image of a first ear (e.g., the right ear) of the patient 102. Then, the robotic arm 116 gradually moves (or rotates) the elliptical mask 114 from second position back to the neutral position.

In some embodiments, the third position may be a second lateral position or a −90 degrees position, and the third position may be closest to the second reflective ball (not illustrated). In aspects, the robotic arm 116 gradually moves (or rotates) the elliptical mask 114 from the neutral position (e.g., zero degrees) to the third position, while continuously capturing images of the face 402 of the patient 102 using the camera(s) 304 and/or the IR sensor(s) 306 embedded in or on the elliptical mask 114. In some embodiments, in the third position, the camera(s) 304 of the elliptical mask 114 is approximately or directly facing and capturing the image of a second ear (e.g., the left ear) of the patient 102. Then, the robotic arm 116 gradually moves (or rotates) the elliptical mask 114 from the third position back to the neutral position.

Therefore, the robotic arm 116 rotates the elliptical mask 114 from i) the neutral position to the first lateral position; ii) the first lateral position to the second lateral position; and iii) the second lateral position back to the neutral position. In some embodiments, the camera(s) 304 and/or the IR sensor(s) 306 of the elliptical mask 114 capture the images of the face 402 and/or a portion of the cranium 112 only during the movement of the elliptical mask 114 from the first lateral position to the second lateral position, which may be approximately 180 degrees. Additionally, or alternatively, during the registration process, the camera(s) 304 and/or the IR sensor(s) 306 of the elliptical mask 114 may capture the images of the face 402 and/or a portion of the cranium 112 during all the movements of the elliptical mask 114 and/or robotic arm 116. In such a case, the elliptical mask 114 may capture the images of the face 402 and/or of the portion of the cranium 112 twice. By so doing, the elliptical mask 114 can capture additional details of the face 402 and/or the cranium 112.

Continuing with step 608, after the elliptical mask 114 captures the images of the face 402 and/or a portion of the cranium 112, the system 100 creates a 3D image target of said portion of the cranium 112. Similar to step 604, to create the 3D image target, the method includes converting the 2D images captured by the elliptical mask 114 into a 3D image (e.g., the 3D image target) using one or more techniques, such as the previously described volume rendering technique.

At step 610, the method may include comparing the 3D image target, which was created when the medical professional initiated the registration process at step 608, to the 3D image source, which was created preoperatively at step 604. In some embodiments, distinct features of the 3D image target represent respective distinct features of the face 402 and/or a portion of the cranium 112, and the distinct features of the 3D image target are included in a first data set. Similarly, distinct features of the 3D image source represent respective distinct features of the cranium 112, and the distinct features of the 3D image source are included in a second data set. The distinct features in both data sets may include dimensions, positions, and/or relative positions of particular parts of the face 402 and/or the cranium 112, as is illustrated and described in the context of FIG. 4 and/or FIG. 5.

In some embodiments, the method may include qualitatively and/or quantitatively comparing the 3D image target to the 3D image source. Specifically, the method may compare the first data set to the second data set by, for example, comparing dimensions, positions, and/or relative positions of each of the distinct features of the 3D image target to the dimensions, positions, and/or relative positions of the same distinct features of the 3D image source. An example of a qualitative comparison between both data sets may include comparing dimensions, positions, and/or relative positions of particular parts of the face 402, such as the eyes, the jaw lines, the zygomatic bones, and/or other parts of the face 402. Another example of a qualitative comparison between both data sets may include comparing dimensions, positions, and/or relative positions of the facial landmarks 404 (e.g., the psychometric points) of the 3D image target to the respective facial landmarks of the 3D image source. Once the method completes the qualitative comparisons with success, the method may map each of the facial landmarks 404 of the 3D image target to each respective distinct feature of the 3D image source.

Additionally, the method may include performing a quantitative comparison of the distinct features, distinct parts, and/or facial landmarks 404 of the 3D image target to the respective distinct features, distinct parts, and/or facial landmarks 404 of the 3D image source. In some embodiments, the method may determine the distance between a first psychometric point to a second psychometric point of the 3D image source. For example, based on the 3D image source, said distance may be 11.5 mm. The method may then determine the distance between the same first psychometric point to the same second psychometric point on the 3D image target. For example, based in the 3D image target, said distance may be 11.6 mm. In some embodiments, although the 3D image target may qualitatively match the 3D image source, if the distances (e.g., measurements, dimensions) are not within a predetermined accuracy threshold (e.g., 95%, 98% accuracy), the method may determine that the 3D image target fails to meet the predetermined standard for registration. If the distances, however, are within the predetermined accuracy threshold, the method may determine that the 3D image target meets the predetermined standard for registration. In such a case, the 3D image target qualitatively and quantitatively matches the 3D image source.

Therefore, at step 612, the method may determine whether the 3D image target matches the 3D image source qualitatively and quantitively. If the 3D image target matches the 3D image source, at step 614, the system 100 or a component thereof (e.g., a speaker, the display screen 502) may inform, or indicate to, the medical professional using an acoustic beep(s); alarm(s); and/or phrases such as “the registration is completed;” “the registration is successful;” “the registration has an accuracy of 99%;” “the registration meets or exceeds the predetermined accuracy threshold;” “the registration meets or exceeds a certain medical standard;” and/or other phrases of such effect. After the method and/or the system 100 informs the medical professional that the registration is successful, the medical professional may start the operation. If the registration is not successful, does not meet a predetermined accuracy threshold, and/or does not meet a certain medical standard, the method can repeat steps 608 to 612. Therefore, some of the steps of the method may be an iterative process.

At step 616, the method determines whether the patient 102's cranium 112, the adjustable three-arm fixture 106, and/or the operating table 104 move after the imaging registration. In some aspects, during the course of the operation procedure, the elliptical mask 114 may remain stationary in the neutral position, and the camera(s) 304 of the elliptical mask 114 may continuously sample and/or monitor the reflective balls of the adjustable three-arm fixture 106. By so doing, the camera(s) 304 of the elliptical mask 114 can detect and quantify movements of the table 104. Similarly, the camera(s) 304 and/or one, some, or all of the IR sensor(s) 306 of the elliptical mask 114 can sample, monitor, and/or quantify any changes in the features and/or the facial landmarks 404 of the face 402 of the patient 102. For instance, the chin of the patient 102 may slightly drop during the operating procedure, even though the cranium 112, the reflective balls, the adjustable three-arm fixture 106, and the table 104 may remain stationary.

At step 618, the method includes performing an autocorrection of the imaging registration. To do so, the method includes utilizing an autocorrection algorithm to account for the change in position by readjusting the registration. The autocorrection algorithm can readjust the 3D image target to match the 3D image source. For example, while the elliptical mask 114 remains stationary in the neutral position, the autocorrection algorithm creates a bounding box around each of the facial landmarks 404 of the face 402 of the patient 102. The dimensions of the bounding boxes may be predetermined to be, for example, 5 mm by 5 mm. Moreover, the autocorrection algorithm determines and tracks the x-y-z coordinates and the centroid of each bounding box. To quantify any movement detected at step 616, at step 618, the autocorrection algorithm computes a Euclidian distance between the x-y-z coordinates and the centroid of each bounding box before the detected movement and the x-y-z coordinates and the centroid of each bounding box after the detected movement. After the autocorrection algorithm quantifies the movement, the autocorrection algorithm updates the x-y-z coordinates of each bounding box to map the movement of any of the facial landmarks 404 of the face 402 and/or the overall movement of the cranium 112. Finally, the autocorrection algorithm and/or the method includes re-registering the x-y-z coordinates to reflect the movement of the facial landmarks 404 of the face 402 and/or the overall movement of the cranium 112. By so doing, the medical professional can obviate the re-initiation of the registration process, and the medical professional can continue the surgery without interruption.

At step 620, the method includes tracking the position(s) of the surgical instruments during the cranial surgical procedure to support and/or provide the cranial surgical procedure navigation. In some embodiments, the robotic arm 116 positions the elliptical mask 114 in the neutral position. The camera(s) 304 of the elliptical mask 114 continuously monitor the reflective balls of the adjustable three-arm fixture 106 to create and/or establish a reference frame for the cranium 112 and the surgical instruments. The IR sensor(s) 306 of the elliptical mask 114 continuously capture the position(s) and/or movement(s) of the surgical instruments.

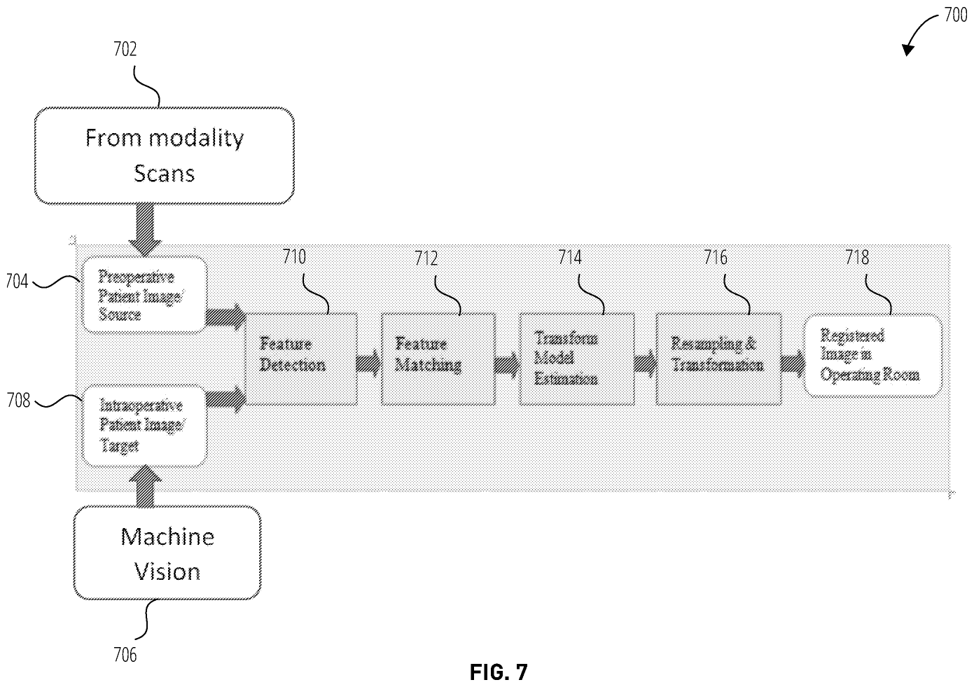

FIG. 7 illustrates a block diagram 700 with additional details of the example method of FIG. 6 for performing the imaging registration, in accordance with examples described herein. FIG. 7 is described in the context of FIGS. 1 to 6. The steps of the method do not necessarily need to be executed in any specific order, or even sequentially, nor need the steps be executed only once, unless otherwise specified. Furthermore, the method can be utilized by using one, more than one, and/or all the steps that are illustrated in FIG. 7. Therefore, the method does not necessarily include a minimum, an optimum, or a maximum number of steps that are needed to implement the systems, methods, and techniques described herein.

Step 702 of the block diagram 700 may be the same as, similar to, and/or equivalent to the step 602 of the block diagram 600. For the sake of clarity, at step 702, the method may include obtaining one or more modality scans of the cranium 112 of the patient 102, where the modality scans may include CT scans, MRI scans, PET scans, SPECT scans, or a combination thereof (e.g., CT-PET scans).

Step 704 of the block diagram 700 may be the same as, similar to, and/or equivalent to the step 604 of the block diagram 600. For the sake of clarity, at step 704, the method may include uploading the modality scans onto the system 100 (or a component thereof) to create a 3D model and/or a 3D image source of the cranium 112. To create the 3D model and/or the 3D image source, the method includes converting the 2D images (e.g., the modality scans) into a 3D image (e.g., the 3D model, the 3D image source) using one or more techniques (e.g., volume rendering techniques). Similar to FIG. 6, the steps 702 and 704 of the block diagram 700 are performed preoperatively.

At step 706, the machine vision system utilizes the camera(s) 304 of the elliptical mask 114 to capture reflected optical (e.g., visible) light off the face 402 and/or a portion of the cranium 112 of the patient 102. As described, for example, in reference to FIG. 3, the camera(s) 304 can convert the reflected light to electrical signals. The resolution of the camera(s) 304 may depend on the working distance, the FOV, the count of physical pixels in the camera(s) 304's sensor, and/or other factors and/or parameters. Furthermore, the camera(s) 304 may be capable of capturing a stereo vision, and the machine vision system can perform 3D measurements having a length, width, and depth perception.

In some embodiments, in addition to the camera(s) 304, the machine vision system may also utilize one, some, or all of the IR sensor(s) 306 to capture RGBIRD images. By so doing, the machine vision system can capture RGB (e.g., color, width, length) information and IRD (e.g., depth) information of the same image frame(s). Consequently, at step 708, the system 100 can convert the 2D images captured by the machine vision system to a 3D image (e.g., the 3D image target, an intraoperative image) of the patient 102's face 402 and/or of a portion of the patient 102's cranium 112 during imaging registration and/or cranial surgical procedure. To create the 3D image target, the system 100 and/or the method of FIGS. 6 and/or 7 may utilize one or more volume rendering techniques.

At step 710, the system 100 can utilize one or more algorithms and/or machine-learned models to detect features (e.g., the facial landmarks 404) of the face 402 in the 3D image target and the 3D image source. The algorithm may be a convolution neural network (CNN) algorithm, a region-based convolutional network (RCNN) algorithm, a region-based fully convolutional network (R-FCN) algorithm, a feedforward neural network (FNN) algorithm, a Harris corner detection algorithm, a Shi-Tomasi corner detector algorithm, a scale-invariant feature transform (SIFT) algorithm, a speeded-up robust features (SURF) algorithm, a binary large object (BLOB) detection algorithm, one or more feature descriptor algorithms, a histogram of oriented gradients (HOG) algorithm, a binary robust independent elementary features (BRIEF) algorithm, and/or a combination thereof and/or another algorithm that can detect the features of the face 402.

At step 712, the system 100 can map the detected features on the 3D image target to the same detected features on the 3D image source. Therefore, at step 712, the system 100 establishes a correspondence among the same features on the 3D image target and the 3D image source. In some embodiments, the correspondence may be established using an intensity distribution in an adjacent area of each pixel in the 3D image target and the 3D image source. The features can be matched based on the similar, the same, or approximately the same measurements and/or dimensions among corresponding anatomical and/or pathological information in the 3D image target and the 3D image source.

At step 714, the system 100 transforms the different sets of data (e.g., the 3D image target, the 3D image source) into a same coordinate system. After the transformation, the system 100 performs the imaging registration. The imaging registration enables the medical professional to perform an IGS. In some embodiments, the imaging registration includes calculating a transformation that maps corresponding points (e.g., psychometric points) between the 3D image target and the 3D image source.

At step 716, the machine vision system resamples the facial landmarks 404 (e.g., the psychometric points) of the face 402 using the machine vision system to create a new 3D image target, and the system 100 transforms again the coordinates of the new 3D image target to the same coordinate system of the 3D image source. In some embodiments, the resampling and the transformation (or re-transformation) of the 3D image target is performed periodically during the cranial surgical procedure. Additionally, or alternatively, the medical professional can initiate the resampling and the transformation. After each resampling and transformation, at step 718, the system 100 performs a registration (or a re-registration) and may calculate the accuracy of said registration. Based on the accuracy of the registration, the system 100 may utilize the autocorrection algorithm to readjust the 3D image target to match the 3D image source.

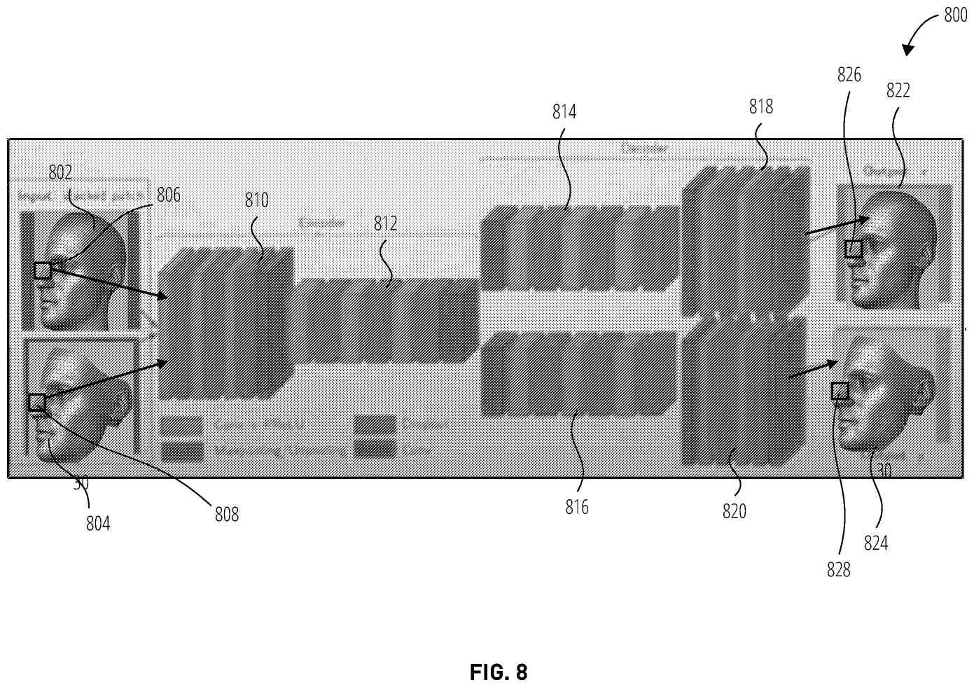

FIG. 8 illustrates a block diagram 800 of an example method for matching features of the 3D image target to the same features of the 3D image source, in accordance with examples described herein. FIG. 8 is described in the context of FIGS. 1 to 7. Furthermore, the method of FIG. 8 includes additional details of the step 712 of FIG. 7. For the sake of brevity, since the example method of FIG. 8 used for matching features of the 3D image target to the same features of the 3D image source includes steps that are prior art, the description of the example method of FIG. 8 is not exhaustive.

Nevertheless, for the sake of clarity, a first input to the system 100 is a 3D image source 802 and a second input to the system 100 is a 3D image target 804. The 3D image source 802 includes a first feature 806, and the 3D image target 804 includes a second feature 808. The first feature 806 and the second feature 808 represent the same feature. In the example illustration of FIG. 8, the first feature 806 and the second feature 808 represent a portion of the patient 102's nose.

In some embodiments, the method of FIG. 8 utilizes a CNN-based encoder-decoder model for extracting the features of the face 402 of the patient 102. The encoder module of the CNN-based encoder-decoder model is illustrated in blocks 810 and 812. Specifically, the encoder module utilizes stacks of at least one convolution network having an activation function (e.g., PReLU) layer, at least one droupout layer, at least one maxpooling/unpooling layer, and/or at least one convolution network layer. The encoder generates maps of the features, such as the maps of the first feature 806 and the second feature 808.

The decoder module of the CNN-based encoder-decoder model is illustrated in blocks 814, 816, 818, and 820 of FIG. 8. In some embodiments, the decoder module also includes at least one convolution network having an activation function (e.g., PReLU) layer, at least one droupout layer, at least one maxpooling/unpooling layer, and/or at least one convolution network layer. The decoder module serves as a classifier of the CNN-based encoder-decoder model for extracted features using the encoder module.

After the encoding and the decoding, the CNN-based encoder-decoder model outputs a reconstructed 3D source image 822 and a reconstructed 3D target image 824. The reconstructed 3D image source 822 includes a first reconstructed feature 826, and the reconstructed 3D image target 824 includes a second reconstructed feature 828. The first reconstructed feature 826 and the second reconstructed feature 828 are the same feature. Moreover, the reconstructed features 826 and 828 are the same as the features 806 and 808.

Skilled persons will appreciate that many changes may be made to the details of the above-described embodiments without departing from the underlying principles of the invention. The scope of the present invention should, therefore, be determined only by the following claims and equivalents.

What is claimed is:

1. A method for performing an imaging registration and a cranial surgical procedure navigation, the method comprising:
obtaining modality scans of a cranium of a patient;
creating, using the modality scans, a first 3D model of the cranium of the patient;
obtaining, using a movable elliptical mask attached to a robotic arm, surface imaging data of a face or a portion of the cranium of the patient while the patient is lying in a supine position atop an operating table, wherein the robotic arm gradually moves the elliptical mask approximately 180 degrees around the face or the portion of the cranium of the patient in a circular motion from:
i) a first position to a second position, wherein the first position comprises the elliptical mask approximately facing a nose of the patient, and the second position comprises the elliptical mask approximately facing an ear of the patient;
ii) the second position to a third position, wherein the third position comprises the elliptical mask approximately facing another ear of the patient; and
iii) the third position to the first position;
creating, using the surface imaging data, a second 3D model of the face or the portion of the cranium of the patient;
registering the second 3D model to the first 3D model; and
guiding a physician during a cranial surgical procedure by displaying the first 3D model, the second 3D model, or a combination thereof on a display screen.

2. A non-transitory computer-readable storage medium including instructions that, when executed by a processor, configure the processor to perform the method of claim 1.

3. The method of claim 1, wherein the modality scans comprise computerized tomography (CT) scans, magnetic resonance imaging (MRI) scans, positron emission tomography (PET) scans, single photon emission computed tomography (SPECT) scans, or a combination thereof.

4. The method of claim 1, wherein:
the modality scans comprise a first plurality of 2D images obtained preoperatively; and
the surface imaging data comprise a second plurality of 2D images obtained during the imaging registration, the cranial surgical procedure navigation, or a combination thereof.

5. The method of claim 1, wherein:
the first position comprises a neutral position of the elliptical mask, and wherein the neutral position comprises a zero-degree movement of the elliptical mask;
the second position comprises a first lateral position of the elliptical mask, and wherein the first lateral position comprises a positive 90 degrees movement of the elliptical mask; and
the third position comprises a second lateral position of the elliptical mask, and wherein the second lateral position comprises a negative 90 degrees movement of the elliptical mask.

6. The method of claim 1, wherein the elliptical mask utilizes a machine vision system to obtain the surface imaging data.

7. The method of claim 6, wherein the machine vision system utilizes at least one imaging sensor, at least one infrared sensor, or a combination thereof to capture red-green-blue (RGB) and infrared (IR) depth (RGBIRD) information of the surface imaging data.

8. The method of claim 1, further comprising tracking positions of instruments during the cranial surgical procedure and presenting positional information of the instruments relative to the first 3D model, the second 3D model, or a combination thereof.

9. The method of claim 1, further comprising:
detecting, using the elliptical mask, a movement of the face, the cranium, the operating table, a fixture holding the cranium, or a combination thereof during the cranial surgical procedure;
quantifying the movement; and
performing an autocorrection of the imaging registration.

10. The method of claim 9, wherein the autocorrection of the imaging registration obviates a need to initiate another registration of the second 3D model to the first 3D model.

11. The method of claim 1, wherein said registering of the second 3D model to the first 3D model meets or exceeds a predetermined accuracy threshold.

12. The method of claim 11, wherein said registering of the second 3D model to the first 3D model further comprises:
detecting features of the face or the portion of the cranium in the first 3D model and the second 3D model; and
mapping the features of the second 3D model to the first 3D model.

13. The method of claim 12, wherein the detection of the features is performed using a convolution neural network (CNN) algorithm, a region-based convolutional network (RCNN) algorithm, a region-based fully convolutional network (R-FCN) algorithm, a feedforward neural network (FNN) algorithm, a Harris corner detection algorithm, a Shi-Tomasi corner detector algorithm, a scale-invariant feature transform (SIFT) algorithm, a speeded-up robust features (SURF) algorithm, a binary large object (BLOB) detection algorithm, one or more feature descriptor algorithms, a histogram of oriented gradients (HOG) algorithm, a binary robust independent elementary features (BRIEF) algorithm, or a combination thereof.

14. The method of claim 1, wherein said creating of the first 3D model and said creating of the second 3D model are performed using the same volume rendering technique.

15. A system for image registration for cranial surgical procedures, the system comprising:
a movable machine vision system mountable to a robotic arm and configured to obtain surface imaging data of a face or a portion of a cranium of a patient;
a fixture holding the cranium, wherein the fixture comprises a first, a second, and a third arm to collectively define a size and a position of the cranium by utilizing respective reflective spheres visible or trackable by the movable machine vision system;
a console configured to:
create a first 3D model of the face or the portion of the cranium using the surface imaging data; and
register the first 3D model to a second 3D model, wherein the second 3D model is created using preoperative medical imaging data; and
a display configured to present to a physician the first 3D model, the second 3D model, or a combination thereof during the cranial surgical procedure.

16. The system of claim 15, wherein the preoperative medical imaging data are obtained using CT scans, MRI scans, PET scans, SPECT scans, or a combination thereof.

17. The system of claim 15, wherein the movable machine vision system is embedded in or on an elliptical mask, and wherein the elliptical mask comprises at least: one camera, one infrared sensor, one light source, or a combination thereof.

18. The system of claim 15, wherein each reflective sphere is mounted at a visible or a trackable end of each arm of the fixture, and each reflective sphere comprises a passive infrared (PIR) sensor used to determine a position of each arm of the fixture from the cranium of the patient.

19. The system of claim 18, wherein each arm of the fixture is adjusted mechanically or electromechanically to fit a size of the cranium using position information determined using each PIR sensor.

20. The system of claim 15, wherein the movable machine vision system tracks positions of instruments during the cranial surgical procedure, and wherein each instrument comprises one or more reflective spheres.
